(19) United States
(12) Patent Application Publication
(10) Pub. No.: US 2016/0331447 A1
(43) Pub. Date: Nov. 17, 2016

(54) METHOD AND APPARATUS FOR SELECTIVE TREATMENT INSIDE A BODY LUMEN

(71) Applicant: THE JOHNS HOPKINS UNIVERSITY, Baltimore, MD (US)

(72) Inventors: Harikrishna Tandri, Ellicott City, MD (US); Menekhem Zviman, Belcamp, MD (US); Ronald David Berger, Baltimore, MD (US)

(21) Appl. No.: 15/106,544

(22) PCT Filed: Dec. 19, 2014
(86) PCT No.: PCT/US2014/071339

§ 371 Date: Jun. 20, 2016

Related U.S. Application Data
(60) Provisional application No. 61/919,139, filed on Dec. 20, 2013.

(51) Int. Cl. A61B 18/14

(52) U.S. Cl. CPC A61B 18/1492; A61B 2018/00029

(57) ABSTRACT

The present invention is directed to device and method for electrically modulating the function of nerves that control sympathetic activity of the renal arteries in the human body. The method includes modifying neural fibers that regulate sympathetic activity of renal tissue to accentuate or attenuate function. The present invention also includes an apparatus for executing methods to regulate renal sympathetic activity via intravascular lumen. Additionally, a system and method to transvenously ablate the renal nerves around the renal artery ostia are disclosed.

### CROSS REFERENCE TO RELATED APPLICATION

This application claims the benefit of U.S. Provisional Patent Application No. 61/919,139 filed on Dec. 20, 2013, which is incorporated by reference, herein, in its entirety.

### FIELD OF THE INVENTION

The present invention relates generally to medical devices. More particularly, the present invention relates to a device and method for treatment of hypertension via regulation of sympathetic outflow to the renal arteries.

### BACKGROUND OF THE INVENTION

Hypertension is a growing clinical problem. It is a leading cause of vascular disease and stroke in the United States and worldwide. Essential hypertension is a multifactorial disorder due to dysregulation of sympathetic tone, increased vascular stiffness, atherosclerosis, abnormal neuroendocrine regulation and failure of renal autoregulation. The majority of subjects often require 2-3 medications for proper control of blood pressure. In some subjects despite several antihypertensive medications, blood pressure control is poor with dire consequence. Poorly controlled hypertension is implicated in development of left ventricular hypertrophy, heart failure, stroke, renal failure and vascular disease.

Many of the drugs that treat hypertension directly or indirectly influence sympathetic tone. Betablockers and ganglion blockers are examples of direct inhibition or blocking of sympathetic hormones, whereas angiotensin converting enzyme inhibitors reduce hypertension by indirectly affecting sympathetic tone.

As such abnormal sympathetic activity is closely connected to the pathogenesis of hypertension. Drugs that reduce sympathetic activity are shown to reduce blood pressure. Surgery directed at reducing abdominal sympathetic tone such as celiac ganglion blockade and resection have been shown to significantly reduce blood pressure over a 2 year follow up period. Recently denervation of the renal arteries has been shown to significantly lower blood pressure in patients with resistant hypertension. Blood pressure reductions of 20 mmHg in systolic blood pressure and 10 mm Hg in diastolic blood pressure have been achieved by radiofrequency energy delivered through a catheter in the renal artery. This procedure is contraindicated in patients who have abnormal tortuous renal vessels and in patients with renal artery stenosis. Renal artery denervation also carries the risk of renal stenosis when ablated close to the ostium and also risk of renal artery dissection.

Histopathology of the renal arteries demonstrates that a majority of the nerves are proximally located close to the origin of the renal arteries from the aorta and decrease in number as the artery enters the renal hilum. The nerves arrive to the renal artery from the ganglia, which are located at variable distance superior to the renal arteries over the aorta, and then spread circumferentially around the renal artery. As such ablation close to the ostium is desired for maximal renal denervation.

Chemical, surgical and radiofrequency ablation of the para vertebral ganglia have been shown to result in hypotension and lowering of sympathetic tone. Although damage to these ganglia can result in damage to the renal sympathetic fibers, these procedures can result in irreversible damage to the sympathetic supply of other organs such as the gut, pancreas and genito-urinary system and hence produce undesirable side effects. Serious side effects such as severe hypotension, gastric dilation, ileus and impotence have been described with ganglionic ablations. Pre aortic ganglia are known to be connected with the renal plexus, inferior mesenteric nerves, celiac and superior mesenteric plexuses, adrenal gland, and possibly with the spermatic and ovarian plexuses through the renal plexus. So a selective method of only ablating the renal nerves while sparing the non-renal nerves is desirable.

US patent application no. 20130165990, 20130165926, 20130165925 describe a balloon with plurality of electrodes deployed in the renal arteries to cause denervation by delivering pulsed electrical energy. The electrodes are non-circumferential and do not achieve complete ostial denervation. Patent application number: 20130116685 discloses a basket within renal vessel with electrodes arranged in a fashion to cause two different circumferential treatment zones inside the renal artery and a transvenous method to access the renal artery to cause electrofusion. Patent application number: 20130012867 discloses a design of an apparatus to cause non-contiguous lesions within the renal artery. Patent application no 20130296836 discloses transaortic ablation of prevertebral ganglia using various energies and patent application number: 20130296443 describes a transvenous method to achieve prevertebral ganglia destruction.

Human studies on the distribution of renal nerves revealed that the total number of nerves are maximum in the proximal renal artery close to the origin from the aorta and also in the anterior region than in other regions. Further, they are localized 2 mm-4 mm within the wall of the renal artery and are much reduced in the endothelium. As such the most attractive region for ablation is the anterior part of the ostium of the renal artery towards the outer wall of the artery.

Accordingly, there is a need in the art for a method and apparatus for simply and reliably accessing the ostium of the renal arteries and effectively denervating the kidneys without the risk of extensive damage to the ganglionic structure or the renal vasculature.

### SUMMARY OF THE INVENTION

The foregoing needs are met by the present invention which provides a method for treating neural tissue from inside a body lumen including inserting an apparatus comprising a catheter having a plurality of electrodes, a radio-frequency energy generator, and a controller into the body lumen. The method also includes delivering electrical energy to a predetermined set of electrodes linearly arranged along the longitudinal axis of the catheter that are in contact with the luminal wall directly adjacent the neural tissue.

In accordance with an aspect of the present invention a method for treating neural tissue from inside a body lumen includes using an apparatus including a catheter having a plurality of electrodes, a radio-frequency energy generator, and a controller. The method also includes delivering electrical energy to a predetermined set of electrodes linearly arranged along the longitudinal axis of the catheter that are in contact with the luminal wall directly adjacent the neural tissue.

In accordance with another aspect of the present invention an apparatus for treating neural tissue from inside a body lumen includes a catheter. The catheter has a plurality of electrodes, a radio-frequency energy generator, and a controller configured to deliver electrical energy to a predetermined set of electrodes linearly arranged along a longitudinal axis of the catheter. The electrodes are in contact with a luminal wall directly adjacent the neural tissue.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1 and 2 illustrate a schematic view of the relationship of the renal veins to the origin of renal arteries.

FIG. 3 illustrates a schematic view of the ganglia on the aorta and the renal nerves that descend on to the renal arteries anterior to the aorta and posterior to the vein.

FIG. 4 illustrates an MRI image of a human subject and the relationship of the origin of the renal arteries and the left renal vein.

FIGS. 5 and 6 illustrate graphical views of the effect of stimulation inside the left renal vein close to the ostia of the renal arteries.

FIG. 7 illustrates graphical views of a similar response by pacing at the ostium of the renal artery through direct arterial cannulation.

FIGS. 8 and 9 illustrate graphical views of the response to stimulation in the renal arteries after ablation at the ostium through transvenous approach. Note significantly blunted response to stimulation post ablation.

FIG. 10 illustrates a schematic view of one of the preferred embodiments of the device to effect renal ostial denervation.

FIG. 11 illustrates a schematic view of another embodiment where the electrodes are arranged linearly on an elongated shaft which has a deflectable mechanism that allows for deployment in the renal vessel.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains, having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to device and method for electrically modulating the function of nerves that control sympathetic activity of the renal arteries in the human body. The method includes modifying neural fibers that regulate sympathetic activity of renal tissue to accentuate or attenuate function. The present invention also includes an apparatus for executing methods to regulate renal sympathetic activity via intravascular lumen. Additionally, a system and method to transvenously ablate the renal nerves around the renal artery ostia are disclosed.

Renal arteries originate at right angles on the side of the aorta below the superior mesenteric artery. The right renal artery is longer than the left as it passes under the inferior vena cava to enter the right kidney. The ostium of the right renal artery lies directly beneath the origin of the left renal vein from the inferior vena cava less than 1 cm right of the midline immediately to the right of the vertebral body. The left renal vein crosses the aorta and lies superior and anterior to the left renal artery approximately 2-3 cm to the left of the midline. This arrangement leads to a predictable and favorable anatomic disposition of the renal artery ostia to the renal vein.

The current apparatus disclosed takes advantage of this reliable anatomy to design a catheter that will reliably engage the ostium of the renal arteries in a region of high renal nerve density to effectively denervate the kidneys. Proximal denervation has been shown to effect degeneration of the nerve distal to the denervated point. Therefore, effective ablation of the proximal nerves might result in a better result than distal non circumferential ablations.

Several methods of denervating the kidneys have been described including ostial renal artery ablations. The majority of these methods have focused on circumferential or linear non circumferential ablation in both renal arteries sequentially to achieve denervation. These procedures often deliver approximately 4-10 watts of radiofrequency energy and significantly debulk the nerve fibers in the renal artery. Both occluding balloons and non-occluding basket mesh arrangements and linear multielectrode catheters arranged in the form of a spiral have been described. None of the above embodiments cause significant ostial denervation of the kidneys. Further, it is required that these catheters individually engage the renal arteries. Also these designs are not useful in the presence of tortuous atherosclerotic renal arteries.

The renal veins on the other hand do not develop atherosclerosis and are rarely tortuous. The left renal vein is a thin walled structure and overlies the origin of both the renal arteries in the aorta making it an attractive and easy option for accessing the renal arteries transvenously. Further the radiologic anatomy is such that the aorta lies approximately 1 cm to the left of the vertebral spinous process and has a diameter of approximately 3-4 cm. The origin of the renal arteries predictably associated with the vertebral process. In an analysis of 30 subjects who underwent a contrast enhanced CT image of the abdomen the ostium of the right renal artery was within 1 cm to the right of the spinous process and the left renal artery was within 2 cm to the left of the spinous process just inferior to the left renal vein. The apparatus in our invention has been specifically designed with electrode spacing and markers to position the catheter to engage both the renal artery ostia while providing excellent contact with the wall of the vein directly adjacent to the neural fibers populating the anterior surface of the renal artery thereby causing targeted ablation with minimal chance of aortic injury.

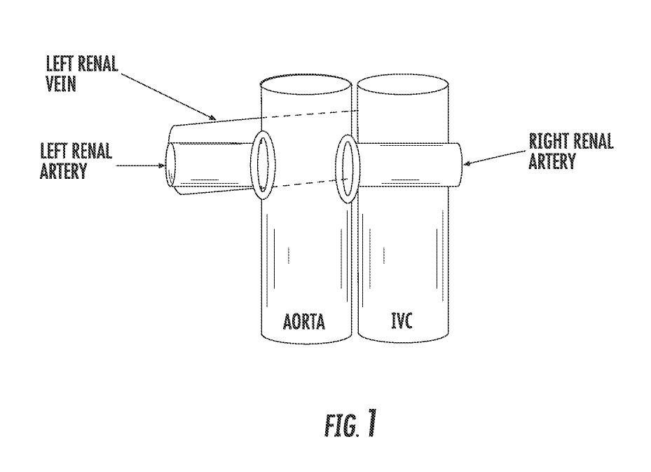

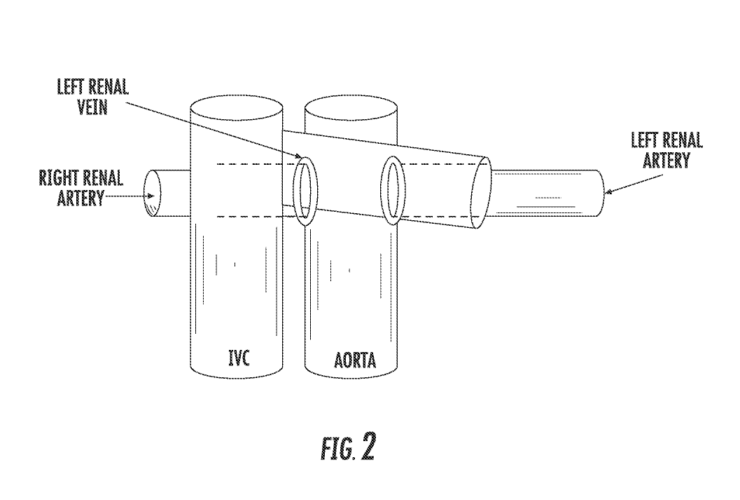

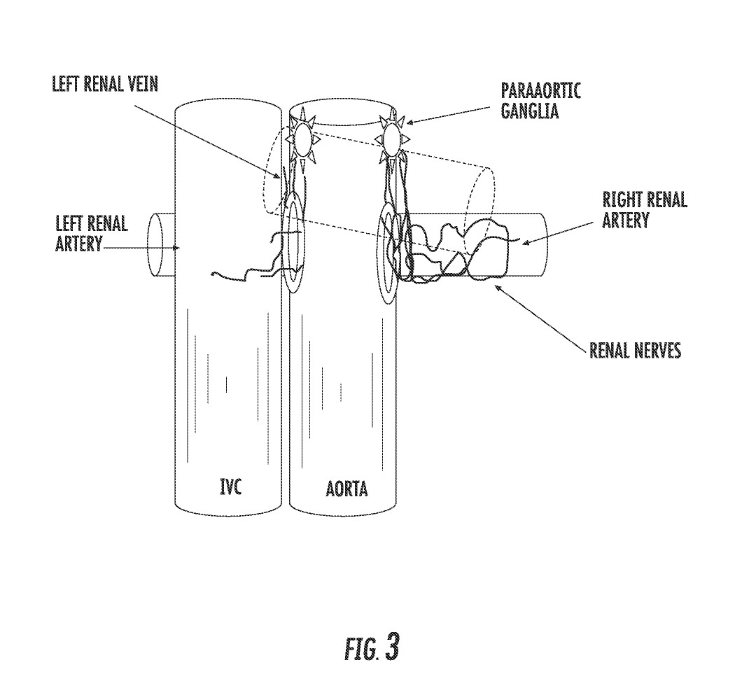

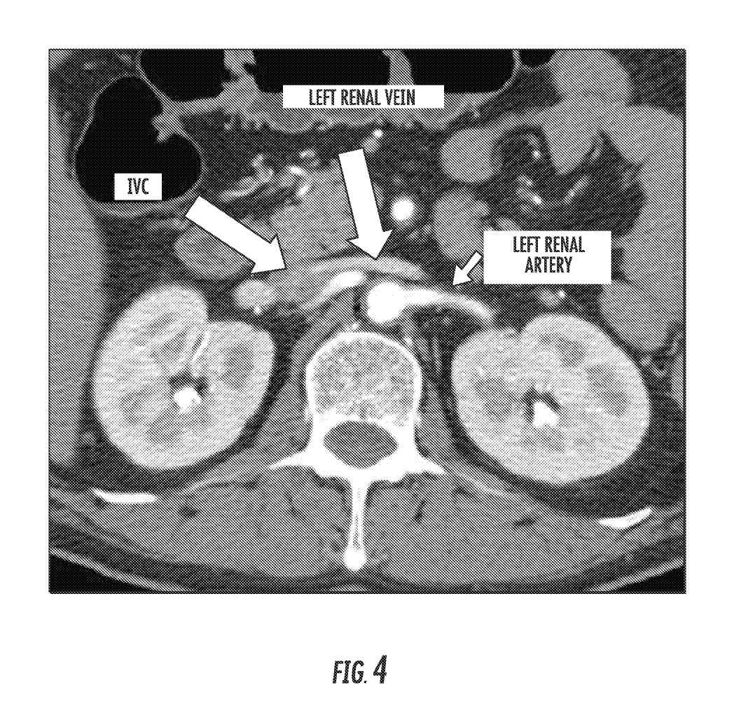

FIGS. 1 and 2 illustrate a schematic view of the relationship of the renal veins to the origin of renal arteries. More particularly, FIG. 1 illustrates a posterior-anterior view of the renal artery ostia and the relationship to the left renal vein. FIG. 2 illustrates an anterior view of the renal artery ostia and the relationship to the left renal vein. FIG. 3 illustrates a schematic view of the ganglia on the aorta and the renal nerves that descend on to the renal arteries anterior to the aorta and posterior to the vein. As illustrated in FIG. 3, the renal nerves from the para aortic ganglia run anterior and enter the renal arteries at the ostia. FIG. 4 illustrates an MRI image of a human subject and the relationship of the origin of the renal arteries and the left renal vein.

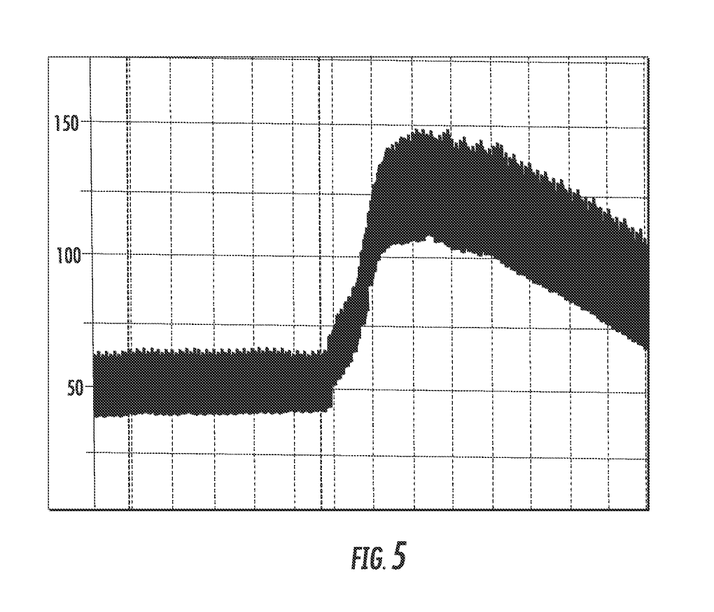

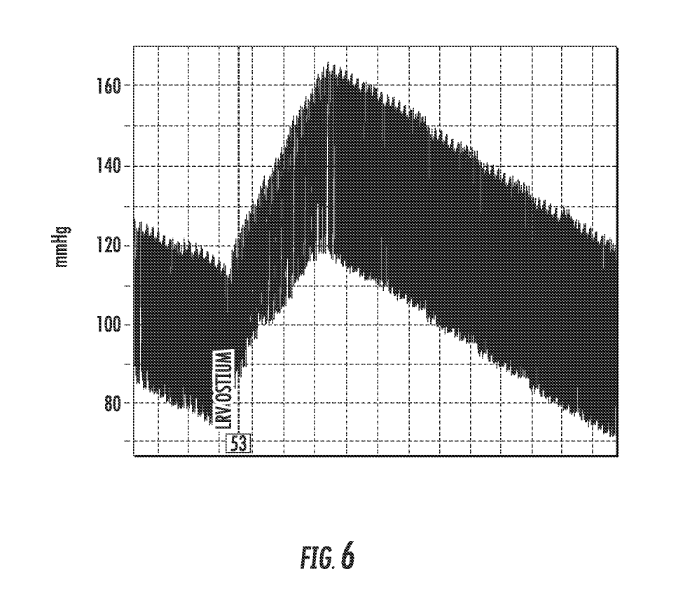

FIGS. 5 and 6 illustrate graphical views of the effect of stimulation inside the left renal vein close to the ostia of the renal arteries. An approximately 20 Hz square wave amplitude of approximately 50 mA with a pulse width of approximately 0.01 sec was used. Note the brisk hypertensive response elicited by pacing at these sites.

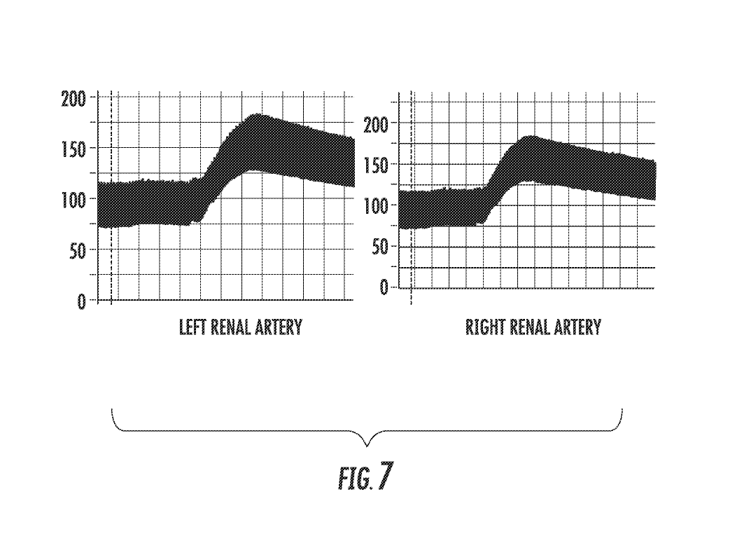

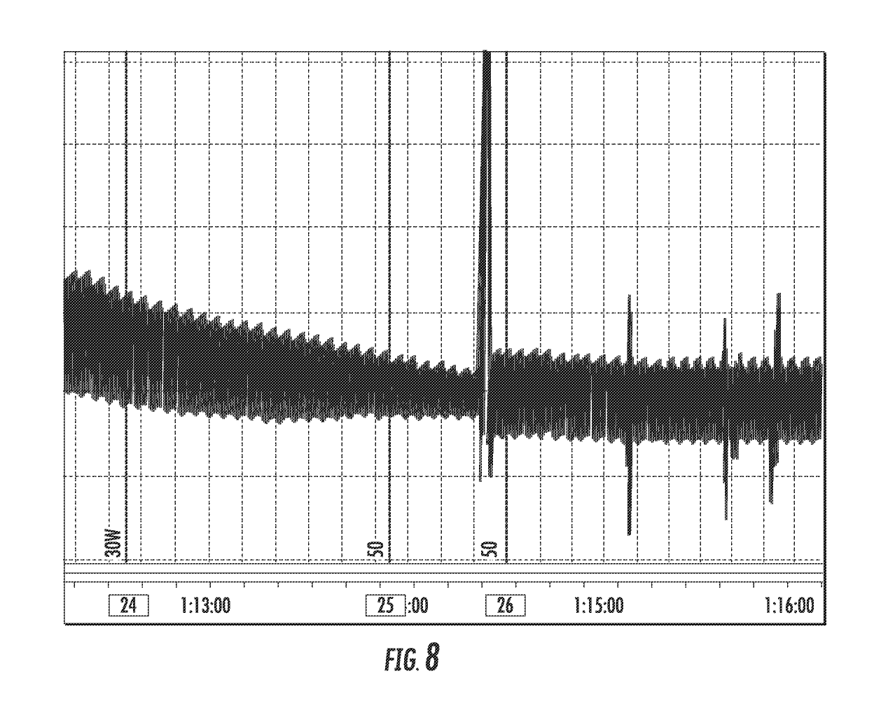

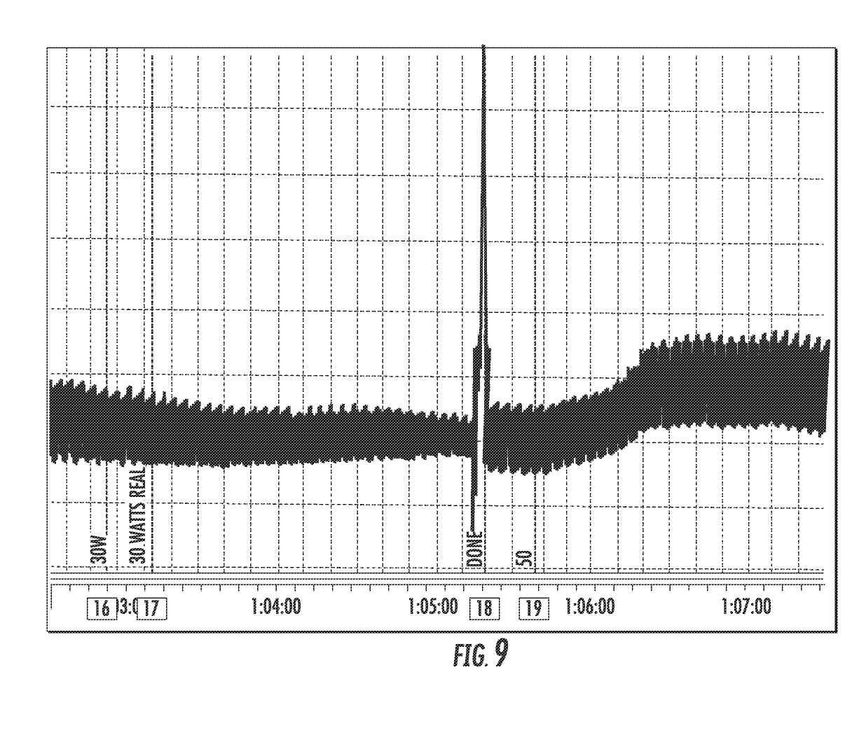

FIG. 7 illustrates a graphical view of a similar response by pacing at the ostium of the renal artery through direct arterial cannulation. FIGS. 8 and 9 illustrate graphical views of the response to stimulation in the renal arteries after ablation at the ostium through transvenous approach. Note significantly blunted response to stimulation post ablation.

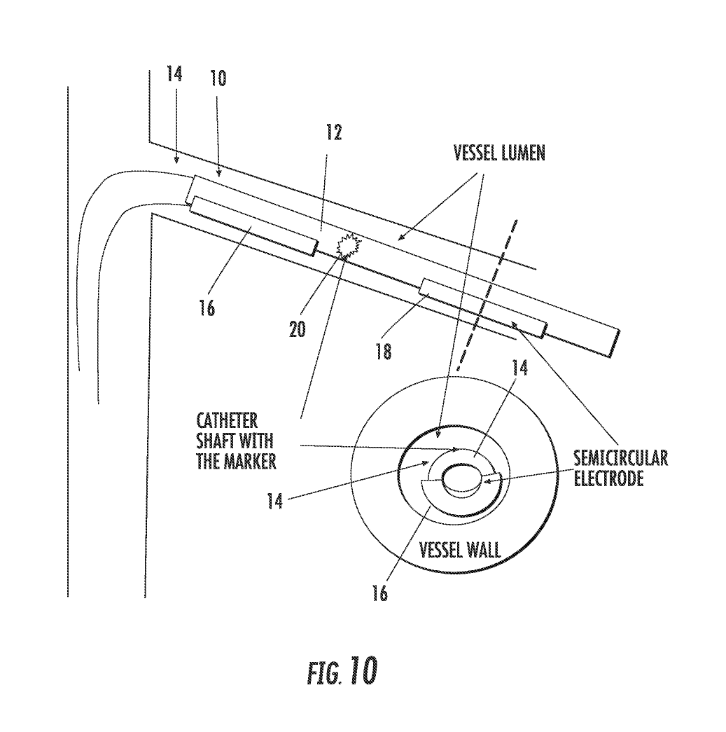

FIG. 10 illustrates a schematic view of one of the preferred embodiments of the device to effect renal ostial denervation. The catheter 12 of the device 10 has an elongated shaft preshaped to engage the renal vessel 14 with two longitudinal electrodes 16, 18, which are approximately 0.2-1 cm long, that are spaced approximately 3 cm apart. A radio opaque marker 20 helps to line up the marker 20 on the catheter 12 to the spinous process thereby placing the two electrodes 16, 18 on the ostia of the renal arteries. The catheter 12 additionally has a central lumen that aids in deploying the catheter 12 within the renal vessel 14. Pulsed electrical energy is delivered in a monopolar fashion simultaneously through both electrodes 16, 18 to effect simultaneous denervation of both renal artery ostia. While two electrodes are shown as an exemplary embodiment in FIG. 10 it should be noted that any number or arrangement of electrodes known to or conceivable by one of skill in the art could also be used.

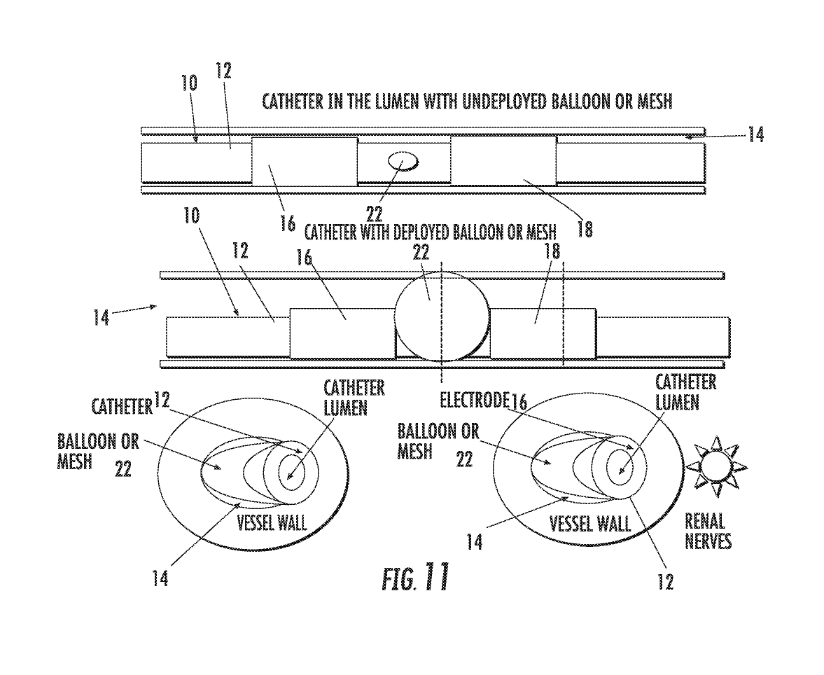

FIG. 11 illustrates a schematic view of another embodiment where the electrodes 16, 18 are arranged linearly on an elongated shaft of the catheter 12 which has a deflectable mechanism that allows for deployment in the renal vessel 14. Following deployment in the inferior vena cava the operator uses the first deflectable mechanism that includes a pull wire to deflect the catheter 12 in to the renal vessel 14. Following placement in the vessel 14 and after moving it to the desired location the operator then uses a fixation mechanism 22 to provide better opposition of the electrodes 16, 18 to the infero-posterior wall of the renal vein directly adjacent to the ostia of the renal artery. The fixation mechanism 22 could include a balloon located between the two ablating electrodes that expands in an eccentric fashion. The balloon distends the vessel and opposes the electrodes to the renal artery ostia thereby avoiding damage to other areas of the renal vein and especially the aorta. Alternatively the fixing mechanism 22 could be a wire mesh that expands eccentrically removing the superior wall of the renal vein away from the ablating electrodes.

The device 10 illustrated in FIG. 11 includes a catheter 12 including electrodes 16, 18, as described with respect to FIG. 10. The device is configured to be disposed within a lumen defined by a wall of the renal vessel 14. The device 10 also includes a balloon or mesh 22 that can be deployed and expanded within the renal vessel 14. The balloon or mesh 22 can be shaped asymmetrically when deployed in order to press one or both of the electrodes against the nerve for ablation.

Another method of fixation includes fixing the electrodes to the wall of the vein directly opposed to the renal vein ostia. The elongated shaft of the catheter has a second pull wire that is positioned in the wall of the catheter in a way that tension on the wire deflects the portion of the catheter between the two electrodes. This when used after deployment in the vessel will shape the catheter in a way that the middle marker portion assumes an inverted U shape and tents the superior wall of the vessel and pushes the electrodes in firm contact with the floor of the renal vein opposite to the origin of the renal artery ostia.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method for treating neural tissue from inside a body lumen comprising:
inserting an apparatus comprising a catheter having a plurality of electrodes, a radio-frequency energy generator, and a controller into the body lumen; and
delivering electrical energy to a predetermined set of electrodes linearly arranged along a longitudinal axis of the catheter wherein the predetermined set of electrodes are in contact with a luminal wall of the body lumen directly adjacent the neural tissue.

2. The method of claim 1 further comprising delivering the electrical energy to renal nerves.

3. The method of claim 1 further comprising inserting the apparatus into the body lumen taking a form of an inferior vena cava or left renal vein or both.

4. The method of claim 1 further comprising inserting the apparatus wherein the apparatus includes a pre biased sheath to cannulate the body lumen.

5. The method of claim 1 further comprising delivering electrical energy to electrodes pre-biased to a shape of a cylindrical basket.

6. The method of claim 1 further comprising delivering electrical energy to an electrode basket that is approximately 1-4 cm long when deployed.

7. The method of claim 6 further comprising dividing the electrode basket into equal quadrants that are electrically isolated from each other and have radio opaque markers to identify the electrode quadrant that is used as a treating electrode.

8. The method of claim 1 further comprising using the apparatus having a central lumen that accommodates a guide wire.

9. The method of claim 1 further comprising including additional lumens in the catheter to allow for irrigant fluid to cool the electrodes.

10. The method of claim 1 further comprising providing the apparatus that can function in a temperature limited or power limited mode.

11. The method of claim 1 further comprising allowing delivery of 4-50 watts of power through the electrodes.

12. A method for treating neural tissue from inside a body lumen comprising:
using an apparatus including a catheter having a plurality of electrodes, a radio-frequency energy generator, and a controller; and
delivering electrical energy to a predetermined set of electrodes linearly arranged along a longitudinal axis of the catheter wherein the predetermined set of electrodes are in contact with a luminal wall of the body lumen directly adjacent the neural tissue.

13. The method of claim 12 further comprising positioning a distal electrode along a distal end of the catheter encircling half of a circumference of the catheter.

14. The method of claim 12 further comprising using an electrode having a 4 mm-20 mm length and generally semicircular in shape.

15. The method of claim 12 further comprising irrigating the electrode to cause surface cooling of tissue below the electrode.

16. The method of claim 12 further comprising positioning a balloon on a distal tip of the catheter diametrically opposite to the electrode to improve the electrode contact with the tissue when deployed.

17. The method of claim 12 further comprising using the catheter having a central lumen for a guide wire.

18. The method of claim 12 further comprising deflecting the catheter in one direction that by design allows for the electrode to face the neural tissue and the balloon to face the opposite vessel wall when inserted in the vessel lumen.

19. The method of claim 12 further comprising delivering the catheter through a deflectable sheath that is predesigned to allow for cannulation of the vessel lumen.

20. The method of claim 12 further comprising delivering with the energy generator high frequency pacing pulses to excite neural tissue to identify targets for ablation.

21. The method of claim 12 further comprising delivering pulses of 3 Hz to 10 k Hz with the energy generator.

22. The method of claim 12 further comprising delivering pulses of variable amplitudes ranging from 5 mA to 1 Ampere using the energy generator.

23. An apparatus for treating neural tissue from inside a body lumen comprising:
a catheter having:
a plurality of electrodes;
a radio-frequency energy generator; and
a controller configured to deliver electrical energy to a predetermined set of electrodes linearly arranged along a longitudinal axis of the catheter,
wherein the electrodes are configured to be in contact with a luminal wall of the body lumen directly adjacent the neural tissue.

24. The apparatus of claim 23 wherein the catheter further comprises a specific shape configured to engage the body lumen in a way that the electrodes align along a particular segment of the body lumen that is adjacent to the neural tissue to be modulated.

25. The apparatus of claim 23 further comprising a stabilizing mechanism that is asymmetric around a primary axis and is within 5 cm of the electrodes wherein the stabilizing mechanism includes a balloon or a wire mesh that when deployed further moves the electrodes to firmly in contact with the luminal wall that is directly adjacent to the neural tissue to be modulated.

26. The apparatus of claim 23 wherein the inter electrode spacing is 4 mm-2 cm.

27. The apparatus of claim 23 further comprising two parallel rows of electrodes on a same side of the catheter shaft each with different inter electrode spacing.

28. The apparatus of claim 23 further comprising a central lumen that accommodates a guide wire.

29. The apparatus of claim 23 further comprising the catheter having a large curvature which when positioned in the lumen aligns the electrodes to the wall adjacent to the neural tissue and the stabilizing mechanism to the wall opposite to the electrodes.

30. The apparatus of claim 23 further comprising the electrodes being circumferential and linearly aligned about the shaft of the catheter.

31. The apparatus of claim 23 wherein the electrodes are irrigated.

32. The apparatus of claim 23 wherein the electrodes are configured for allowing delivery of pulsed electrical energy with a power of 4-40 watts.

33. The apparatus of claim 25 wherein the stabilizing mechanism is a jet of irrigant fluid that is delivered to the lumen diametrically opposite to the electrodes to improve contact of electrode to the lumen adjacent to neural tissue and avoid damage to the opposite wall during pulsed electrical ablation.

34. An apparatus for treating neural tissue from inside a body lumen comprising:
a catheter pre-shaped to engage a left renal vein, having a plurality of electrodes linearly arranged along a longitudinal axis of the catheter, wherein said catheter has a stabilizing mechanism that is asymmetric around the primary axis and is within 5 cm of said electrodes, and wherein said electrodes are preferentially arranged on the catheter opposite the side of said stabilizing mechanism;
a radio-frequency energy generator; and
a controller to deliver electrical energy to a predetermined set of said electrodes.

35. A method of transvenously ablating nerves of both kidneys of a patient through a single placement of a catheter in a left renal vein of the patient comprising:
placing an energy delivery catheter in a left renal vein with an energy delivering element of the catheter abutting therapy zone of the posterior wall of the left renal vein between ostium and 5 cm distal from the ostium in to the left renal vein; and
delivering ablative thermal energy to the selected zone to create one or more lesions along the posterior surface of vein in the selected treatment zone.
